(19) United States
(12) Patent Application Publication
(10) Pub. No.: US 2026/0155215 A1
(43) Pub. Date: Jun. 4, 2026

(54) CHARACTERIZING AND EVALUATING PRE-TRAINED AND TASK-ADAPTED DEEP LEARNING REPRESENTATIONS

(71) Applicant: INTERNATIONAL BUSINESS MACHINES CORPORATION, ARMONK, NY (US)

(72) Inventors: Celia Cintas, Nairobi (KE); Payel Das, Yorktown Heights, NY (US); Jarret Ross, Wichita, KS (US); Skyler Speakman, Nairobi (KE)

(21) Appl. No.: 18/967,580

(22) Filed: Dec. 3, 2024

(51) Int. Cl. G16C 20/30; G16C 20/70

(52) U.S. Cl. CPC G16C 20/30; G16C 20/70

(57) ABSTRACT

A system, method and computer program code are described that provide a computer-implemented method for characterizing and evaluating a pre-trained model. The method includes training an unsupervised model using non-parametric property-driven subset scanning to provide a characterization of embeddings of the pre-trained model and using the characterizations to compute a metric to contrast different domain adaptation methods for the pre-trained model.

### BACKGROUND

The present disclosure generally relates to systems and methods for characterizing and evaluating pre-trained and task-adapted deep learning representations, and more particularly, to systems and methods for localizing and characterizing representations of pre-trained models through the lens of non-parametric property-driven subset scanning (PDSS) in order to improve the interpretability of deep molecular representations.

With the surge of pre-trained deep learning models (also known as Foundational Models), the question of which representation is best for deployment is gaining importance to both machine learning (ML) researchers and practitioners, as well as which parts of the representation model are responsible or needed for a given downstream task.

There is a wide range of methods for this purpose, such as mutual information between representations and labels, feature projection to new target domains, mechanistic interpretability, and probing. Most of the proposed frameworks for evaluation are heavily dependent on the type of model, tasks, and adaptation technique used.

Pre-trained deep learning (DL) models are also rapidly emerging as tools for enhancing scientific workflow and accelerating scientific discovery. For example, representation learning is fundamental in studying the molecular structure-property relationship, which is then leveraged to predict molecular properties or design new molecules with desired attributes. Given the complexity of molecular structure-function relationships, a plethora of DL models have emerged that take in text-based annotations, graphs, and 3D structure as input. Recently, self-supervised learning methods for molecular representation have been employed to address insufficient labeled molecules and learn a task-agnostic universal representation. The pre-trained molecular models are diverse in nature, vary in size and architecture, are trained using particular self- or un-supervised methods, or are domain-adapted via task-specific finetuning. While these models have improved performance for generative and predictive benchmarks, the semantics of the learned representations remain opaque.

### SUMMARY

A system, method and computer program code are described that provide a computer-implemented method for characterizing and evaluating a pre-trained model. The method includes training an unsupervised model using non-parametric property-driven subset scanning to provide a characterization of embeddings of the pre-trained model and using the characterizations to compute a metric to contrast different domain adaptation methods for the pre-trained model.

These and other features will become apparent from the following detailed description of illustrative embodiments thereof, which is to be read in connection with the accompanying drawings.

### BRIEF DESCRIPTION OF THE DRAWINGS

The drawings are of illustrative embodiments. They do not illustrate all embodiments. Other embodiments may be used in addition or instead. Details that may be apparent or unnecessary may be omitted to save space or for more effective illustration. Some embodiments may be practiced with additional components or steps and/or without all the components or steps that are illustrated. When the same numeral appears in different drawings, it refers to the same or like components or steps.

FIG. 1 shows an overview of a framework illustrating two scenarios for inspecting a molecular embedding from chemical language models (CLMs), consistent with an illustrative embodiment;

FIG. 2A shows score distributions for group property-driven subset scanning over fine-tuned and task-agnostic MoLFormer embeddings, illustrating how the detection power improves when scanning the fine-tuned embeddings compared to the task-agnostic version for the inhibitory binding of B-secretase (BACE) binary classification task, consistent with an illustrative embodiment;

FIG. 2B shows score distributions for group property-driven subset scanning over fine-tuned and task-agnostic MoLFormer embeddings, illustrating how the detection power improves when scanning the fine-tuned embeddings compared to the task-agnostic version for the blook-brain barrier penetration (BBBP) binary classification task, consistent with an illustrative embodiment;

FIG. 2C shows score distributions for group property-driven subset scanning over fine-tuned and task-agnostic MoLFormer embeddings, illustrating how the detection power improves when scanning the fine-tuned embeddings compared to the task-agnostic version for the inhibition of human immunodeficiency virus (HIV) binary classification task, consistent with an illustrative embodiment;

FIG. 3 shows that HIV and BACE, that both involve enzyme inhibition, share almost double the elements as compared to the ˜13 nodes shared with BBBP, which is associated with a fundamentally different mechanism;

FIG. 4A shows a cardinality distribution of detected elements for BBBP with task-agnostic embeddings;

FIG. 4B shows a cardinality distribution of detected elements for BBBP with fine-tuned embeddings;

FIG. 5 shows shared elements of pre-trained embeddings between no-flavor, bitter, and sweet found through the group property driven subset scanning method, consistent with an illustrative embodiment;

FIG. 6 shows shared elements of pre-trained embeddings between no-flavor, bitter, and sweet found through the group property driven subset scanning method, consistent with an illustrative embodiment;

FIG. 7 shows score distributions (SD) for group of elements in l_{Σ} that contain representations that have the properties described in Multi-level Performance Evaluation of Generative mOdels (MPEGO) Ruleset, where better characterization capabilities are observed in l_{Σ} extracted from GraphAF;

FIG. 8 shows a table illustrating a description of the base models used to extract pre-trained, fine-tuned, and projected embeddings from CLMs and summarization layers from AGGMs, type of the alternative hypothesis defined in each experiment (H₁), the number (#) of samples used to build the null hypothesis (H₀), and to test H₁ in the different scenarios;

FIG. 9 shows a table of PDSS averaged detection power (in AUC) across five runs for tasks under the scenario large (n>1000) and small sample sizes in the target task (n=100) across both pre-trained embeddings MoLFormer and ChemBERTa, where (*) indicates the sample size for the BACE task and (**) indicates the sample size for the BBBP task;

FIG. 10 shows a table of detection power (AUC) across two different Generative Models (AGGMs)-GraphAF and GCPN, trained in two datasets, ZINC250k and MOSES, where the first H₁ corresponds to finding the graph inner representation of valid molecules, the second H₁ corresponds to a group of molecules with a specific set of chemistry properties, and NA designates a ruleset is not available for that dataset;

FIG. 11 shows a table of classification with different features as inputs—first, using all elements from the pre-trained embeddings, second, using a random selection of elements from the same embeddings, and lastly, using the union of subset found in an unsupervised manner by the PDSS method, consistent with an illustrative embodiment; and

FIG. 12 is a functional block diagram illustration of a computer hardware platform that can be used to implement the method for characterizing and evaluating a pre-trained model, consistent with an illustrative embodiment.

### DETAILED DESCRIPTION

In the following detailed description, numerous specific details are set forth by way of examples to provide a thorough understanding of the relevant teachings. However, it should be apparent that the present teachings may be practiced without such details. In other instances, well-known methods, procedures, components, and/or circuitry have been described at a relatively high-level, without detail, to avoid unnecessarily obscuring aspects of the present teachings.

As described in greater detail below, aspects of the present disclosure provide systems and methods that can determine a linguistic boundary condition for synthetic data generation in a multi-class classification problem.

According to an aspect of the present disclosure, there is provided a computer-implemented method, a system and a computer program product for characterizing and evaluating a pre-trained model. The method includes training an unsupervised model using non-parametric property-driven subset scanning to provide a characterization of embeddings of the pre-trained model and using the characterizations to compute a metric to contrast different domain adaptation methods for the pre-trained model.

In an embodiment, which can be combined with the preceding embodiment, the method further includes using the non-parametric property-driven subset scanning methods on language embeddings and node activations from summarization layers of the pre-trained model.

In some embodiments, which can be combined with any of the preceding embodiments, the method further includes reducing a space of subsets through a linear-time subset scanning property while identifying a subset with a highest score.

In some embodiments, which can be combined with any of the preceding embodiments, the metric is computed for a given task unseen by the pre-trained model.

In some embodiments, which can be combined with any of the preceding embodiments, the training of the unsupervised model includes forming a distribution of expected activations at each element of an embedding vector; scoring a group of samples in a test set by recording the embeddings induced by the group of test samples and comparing them to baseline embeddings created from the distribution of expected expectations to provide a p-value for each sample in the test set at each embedding; and measuring a degree of anomalousness of each p-value.

In some embodiments, which can be combined with any of the preceding embodiments, the method further includes using the characterizations to determine a wellness of the pre-trained model for a new task.

In some embodiments, which can be combined with any of the preceding embodiments, the method further includes using the characterizations as element localization within the embeddings.

In some embodiments, which can be combined with any of the preceding embodiments, the method further includes receiving an input regarding computational constraints for running the pre-trained model, and outputting a combination of the pre-trained model and adaptation techniques best for a new task and the computational constraints.

In some embodiments, which can be combined with any of the preceding embodiments, a computer-implemented method for characterizing and evaluating multiple pre-trained models includes training an unsupervised model using non-parametric property-driven subset scanning to provide a characterization of embeddings of each of the pre-trained models. The characterizations are used to compute a metric to rank each of the pre-trained models for a new task. The method can further receive an input regarding computational constraints for running the pre-trained model and output a combination of one of the pre-trained models and an adaptation technique best for the new task and the computational constraints.

Although the operational/functional descriptions described herein may be understandable by the human mind, they are not abstract ideas of the operations/functions divorced from computational implementation of those operations/functions. Rather, the operations/functions represent a specification for an appropriately configured computing device. As discussed in detail below, the operational/functional language is to be read in its proper technological context, i.e., as concrete specifications for physical implementations.

Accordingly, one or more of the methodologies discussed herein may characterize and evaluate the internal representation of pre-trained models to better inform data efficiency and sampling, robustness, and interoperability. This may have the technical effect of providing a metric to contrast different domain adaptation methods as well as quality assessment of generative processes to provide optimally adapted embeddings for a given task. Accordingly, the system and methods according to embodiments of the present disclosure provide a substantial improvement to technology and computer functionality.

It should be appreciated that aspects of the teachings herein are beyond the capability of a human mind. It should also be appreciated that the various embodiments of the subject disclosure described herein can include information that is impossible to obtain manually by an entity, such as a human user. For example, the type, amount, and/or variety of information included in performing the process discussed herein can be more complex than information that could be reasonably be processed manually by a human user.

Embodiments of the present disclosure can provide systems and methods to characterize and evaluate the internal representation of pre-trained models to better inform data efficiency and sampling, robustness, and interoperability. The methods include training an unsupervised method to characterize embeddings of pre-trained models, then using found subset of elements as a metric to intelligibly select the best adapted embedding for a given task or new domain.

Molecular representations can be characterized to (1) determine which pre-trained representation is more task-optimal, (2) evaluate if and to what extent an adaptation method is needed for a pre-trained representation (and which approach among fine-tuning, projection, or the like, will work best for a new task), and (3) enable more fine-grained introspection in molecular generation (e.g., a user might want to generate molecules with a logical combination of multiple properties, e.g., absence of scaffold AND molecular weight<=270 OR $>=550 Daltons AND Log P>=1.3). To this end, mapping multiple, rather than a single property, to the activation space before generation is beneficial.

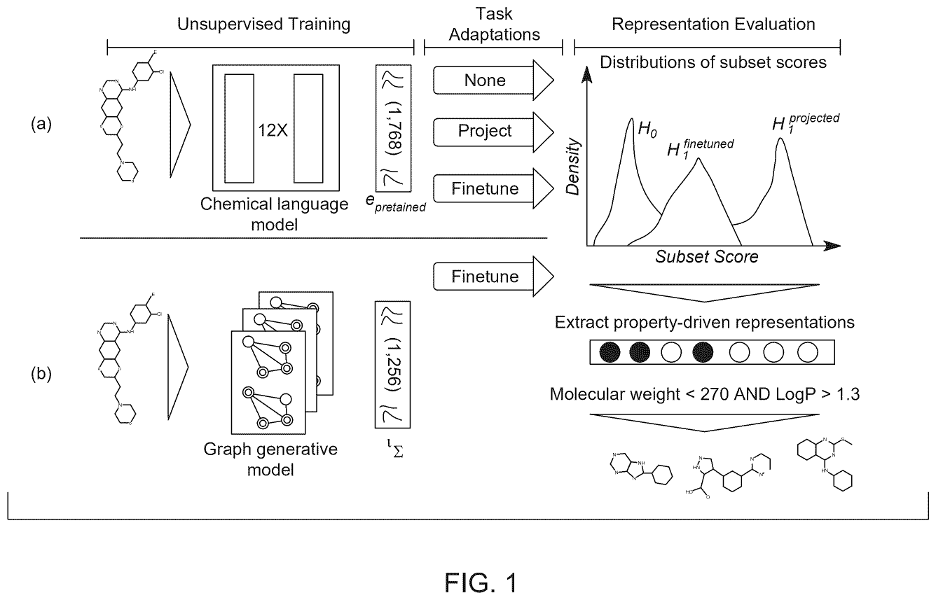

In FIG. 1, an overview of the proposed framework is illustrated. Two scenarios are shown for inspecting a molecular embedding from chemical language Models (CLMs) (in row (a)) and graph generative models (in row (b)). First, the distribution of the summarization layer l_{Σ} of a graph generative model, or an embedding e_{pre-trained} from a CLM architecture, is analyzed. After the elements are extracted, the empirical p-values are computed, followed by the maximization of non-parametric scan statistics. Finally, distributions of subset scores expected candidates and alternative ones are estimated, and a subset of candidates with the corresponding identified subset of elements that contributed to that score.

According to embodiments of the present disclosure, subset scanning methodologies are extended for characterization and evaluation of the quality of molecular representations. This has the potential to provide a metric to contrast different domain adaptation methods as well as quality assessment of generative processes.

Group property-driven subset scanning uses non-parametric scan statistics (NPSS). Given that NPSS makes minimal assumptions on the underlying distribution of node activations, the approach, according to embodiments of the present disclosure, has the ability to scan across different types of embeddings, layers, and activation functions, as described herein.

There are three steps to using the non-parametric scan statistics on the model's activations. The first step is an expectation step, forming a distribution of “expected” activations at each node (H₀). This distribution is generated by letting the generative process create samples that are known to be from the training data, sometimes referred to as “background” samples, and the activations at each node are recorded. The second step is a scoring step, scoring a group of samples in a test set that may contain candidates with a given property or not. The activations induced by the group of test samples are recorded and compared to the baseline activations created in the first step. This comparison results in a p-value for each sample in the test set at each node. The third step is a quantification step, where the degree of anomalousness of the resulting p-values is measured by finding Xₛ and Oₛ that maximize the NPSS, which estimates how much an observed distribution of p-values deviates from the uniform distribution.

As described in greater detail below, aspects of the present disclosure provide (1) an unsupervised approach to detect and characterize pre-trained molecular representations. Two emerging and distinct classes of off-the-shelf models are considered herein: (a) chemical language foundation models, and (b) autoregressive chemical graph generative models. Group property-driven subset scanning (PDSS) methods are applied on language embeddings and node activations from summarization layers; (2) the ability to compare pre-trained, fine-tuned, and projected representations for a given task that is unseen by the model. The effect of identified elements in the different representations are analyzed. This reveals notable information condensation in the pre-trained embeddings upon task-specific finetuning; and (3) an evaluation of the methods of the present disclosure with detection power metrics across multiple datasets, generative models, domain adaptation techniques, and task granularity, from simple validity tests, to molecules with a fine-grained ruleset of properties.

### Scanning Over Molecular Representations

Aspects of the present disclosure extend subset scanning methodologies to characterization and evaluation of the quality of molecular representations has the potential to provide a metric to contrast different domain adaptation methods as well as quality assessment of generative processes.

A visual overview of the proposed characterization framework and practical scenarios is shown in FIG. 1. Consider a set of samples from the embedding vectors X={X₁ . . . X_{M}} and elements O={O₁ . . . O_{J}} generated e.g., by CLM_{Encoder}, where CLM_{Encoder} is a chemical language model encoder capable of producing task-agnostic and fine-tuned molecular embeddings. Let Xₛ ⊆X and Oₛ⊆O, and then define the subsets S under consideration to be S=Xₛ×Oₛ. The goal is to find the most property-driven subset:

$${S}^{*} = arg {max}_{S} F ( S ) \qquad ( 1 )$$

where the score function F(S) defines the anomalousness of a subset of samples from the elements of a given component from CLM_{Encoder} or a graph generative model (AGGM). Group property-driven subset scanning uses an iterative ascent procedure that alternates between two steps: a step identifying the most property-driven subset of samples for a fixed subset of elements, or a step that identifies the converse. There are 2M-1 possible subsets of samples, Xₛ, to consider at these steps. However, the linear-time subset scanning property (LTSS) reduces this space to only M possible subsets while still guaranteeing that the highest scoring subset will be identified. This drastic reduction in the search space is the feature that enables subset scanning to scale to large networks and sets of samples.

### Non-Parametric Scan Statistics

Group property-driven subset scanning uses non-parametric scan statistics (NPSS) that makes minimal assumptions on the underlying distribution of node activations. Aspects of the present disclosure has the ability to scan across different types of embeddings, layers, and activation functions, as described herein.

As briefly discussed above, there are three steps to using the non-parametric scan statistics on the model's activations. First is an expectation step, forming a distribution of “expected” activations at each node (H₀). This distribution is generated by letting the generative process create samples that are known to be from the training data, sometimes referred to as “background” samples, and record the activations at each node.

The second step is a scoring step, scoring a group of samples in a test set that may contain candidates with a given property or not. The activations induced by the group of test samples are recorded and compared to the baseline activations created in the first step. This comparison results in a p-value for each sample in the test set at each node.

The third step is a quantification step, where the degree of anomalousness of the resulting p-values are measured by finding Xₛ and Oₛ that maximize the NPSS, which estimates how much an observed distribution of p-values deviates from the uniform distribution.

Group-based subset scanning computes an empirical p-value for each node, as a measurement of how divergent the activation (l_{Σ}) or embedding (e) value of a potentially novel sample is at a given node. This p-value estimates the proportion of activations from the background samples, that are larger or equal to the activation from an evaluation sample. Group-based subset scanning processes the matrix of p-values (P) from test samples with NPSS to identify a submatrix S=Xₛ×Oₛ that maximizes F(S), as this is the subset with the most statistical evidence for having been affected by a property-driven pattern. The general form of the NPSS score function is

$$F ( S ) = {max}_{α} {F}_{α} ( S ) = {max}_{α} ϕ ( α , {N}_{α} ( S ) , ( S ) ) \qquad ( 2 )$$

where N(S) is the number of empirical p-values contained in subset S and N_{α}(S) is the number of p-values less than (significance level) α∈(0, 1) contained in subset S. It has been shown that for a subset S consisting of N(S) empirical p-values, it holds: E [N_{α}(S)]=N(S)α. Group-based subset scanning attempts to find the subset S that shows the most evidence of an observed significantly higher than an expected significance, N_{α}(S)>αN(S), for significance level α. In this work, the higher-criticism test is used as a scan statistic. This can be interpreted as the test statistic of a Wald test for the amount of significant p-values given that N_{α} is binomially distributed with parameters N_{α} and α.

$$\frac{\vert {N}_{α} - N α \vert}{\sqrt{N α ( 1 - α )}} \qquad ( 3 )$$

Because higher-criticism normalizes by the standard deviation of Na, it tends to be more sensitive to small subsets with very extreme p-value ranges as this would produce large values in the numerator and smaller ones in the denominator.

### Experimental Setup

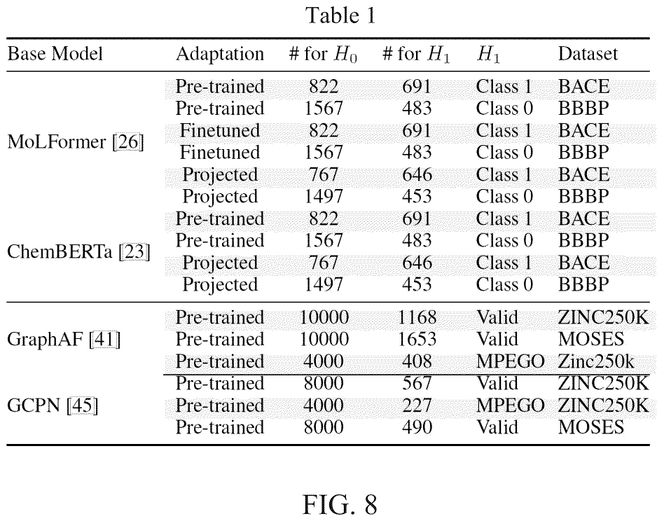

Extensive experiments were performed to validate the generalizability and characterization capabilities of the systems and methods according to embodiments of the present disclosure. To this end, two groups of models were evaluated: Autoregressive Graph Generative Models (AGGMs) and Chemical Language Models (CLMs). Models in each group were evaluated across multiple datasets, domain adaptation techniques, and downstream tasks (see Table 1, represented in FIG. 8).

The set-up details employed in each experimental validation is provided. All group property-driven subset scanning experiments presented herein were performed in a desktop machine (2.9 GHZ Quad-Core Intel Core i7, 16 GB 2133 MHz LPDDR3). All models, AGGMs, and CLMs, were off-the-shelf trained models.

### Representation Analysis in Chemical Language Models

The quality of molecular embeddings obtained from different CLMs and domain adaptation techniques were assessed. Particularly, task-agnostic, fine-tuned representations obtained from MoLFormer and ChemBERTa were examined. For both CLMs, publicly available pre-trained and fine-tuned models were used. Projection methods, such as Pro2 [8] were evaluated. These projection methods learn a projection that maps pre-trained embeddings onto orthogonal directions and learn a classifier using projected embeddings in limited target data settings. To find an optimal number of features for the projection, a grid search of 10 to 200 elements was performed for each task. Two molecular property prediction tasks from MoleculeNet were employed as the downstream tasks to evaluate the representations from CLMs.

These tasks are binary classification tasks: BACE (binding results for a set of inhibitors of human β-secretase, (n=1522)), and BBBP: Blood-brain barrier permeability, (n=2000). A smaller target set of n=100 of each previously mentioned task was used to compare projection features and finetune representations.

### Representation Analysis in Autoregressive Graph Generative Models

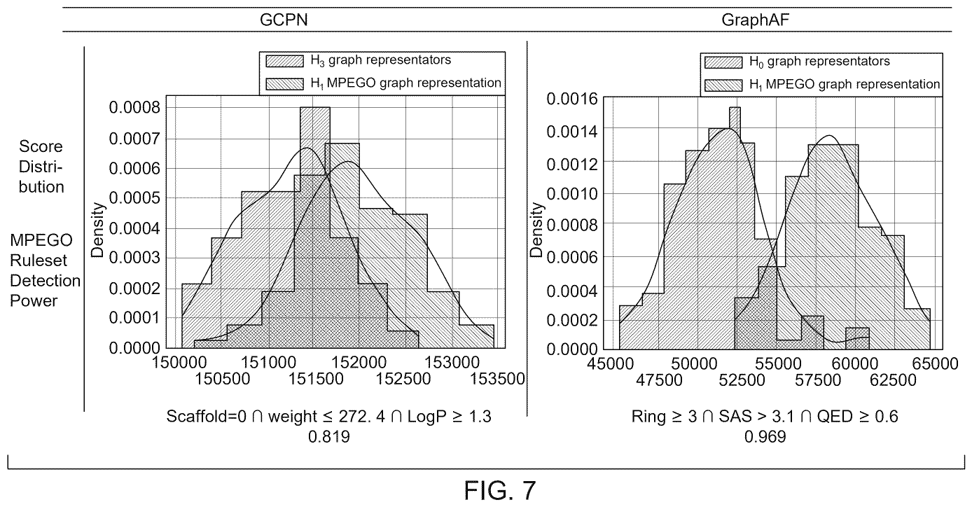

Autoregressive Graph Generative Models (AGGMs) were employed to further evaluate the capability of the methods according to embodiments of the present disclosure in providing fine-grained control in the generation process. Specifically, two AGGMs were used, (1) Graph Convolutional Policy Network (GCPN) and (2) a Flow-based Autoregressive (GraphAF) as the graph generator. GCPN employed a reinforcement learning strategy for molecular graph generation that optimized domain-specific characteristics through policy gradient. GraphAF aimed to exploit the advantages offered by both autoregressive and flow-based approaches in order to provide enhanced flexibility, efficiency, and improved sampling process to encode domain knowledge. Both GCPN and GraphAF were trained on the publicly available ZINC-250K dataset, which contains 249,455 small molecules. Additionally, a refined version of ZINC4 molecules was used, as proposed by the benchmark platform MOSES, which undergoes filtering by certain parameters, such as molecular weight ranges and the number of rotatable bonds, among others. For both GCPN and GraphAF models, scanning was performed over a summarization layer (l_{Σ}), which concatenates node and edge representation. In this case, the learned representation was evaluated on two different tasks. First, the detection of invalid graphs in the learned representation space (lg), and the second task, is to identify candidates with a given set of rules generated by MPEGO. An example of Rulesets generated by MPEGO for each graph generative model and dataset can be seen in FIG. 7. These sets of properties correspond to molecules being generated with higher or lower frequencies.

### Metrics

The area under the receiver operating characteristic curve (AUC) and precision (P) were employed as performance metrics in both generation and representation analyses. In group property-driven scanning results, AUC can be thought of as detection power, which is the method's ability to distinguish between test sets that contain some proportion of molecule candidates from H₁ and test sets containing only samples from H₀. P reflects detection performance, which is the method's ability to label which candidates in the test set belong to H₁. Table 1 in FIG. 8, which details regarding H₀ and H₁ for each experiment.

### Baseline and Scanning Setup

The methods of the present disclosure was compared with an existing baseline, which was proposed for a simpler characterization of inner representations in neural networks, but without encoding group-level characteristics. The framework according to embodiments of the present disclosure utilizes group property for the characterization of node activations extracted from task-agnostic and fine-tuned embeddings (|e|=768) generated by MoLFormer and ChemBERTa, as well as summarization layers (|l_{Σ}|=256) from GCPN and GraphAF.

One assumption of the experiments described herein is how the null hypothesis or expectation (H₀) is defined. Table 1, in FIG. 8, shows the details of the experiments, including a description of the alternative hypotheses (H₁) and sample sizes used to build the hypotheses. H₀ was extracted from a forward pass of the known data through the encoder of CLMs (CLM_{Encoder}) or a AGGM and record the activations at each node. For the downstream tasks such as BACE and BBBP, H₀ contains the most common class and H₁ will only have the remaining class. In the validity test, H₀ is designed to contain only invalid graph representations; these are generated at a higher rate in both generative graph generative models. In the MPEGO ruleset case, H₀ will contain valid representations that do not contain properties found in the ruleset. The H₁ distributions are built from a forward pass with only samples belonging to a given class (BACE and BBBP downstream tasks), valid representations (for the validity use-case), and representations that generate molecules with a given set of properties for the MPEGO use-case. During testing, 100 randomized runs were set. In each run, test sets were created with samples from both H₀ and H₁ to assert the detection capabilities. The threshold αₘₐₓ was set to 0.5, a search parameter in the methods according to aspects of the present disclosure. It is possible to tune αₘₐₓ to increase detection further for each particular downstream task in a supervised manner.

### Results

This section describes the results of the methods according to embodiments of the present disclosure across the two main experimental platforms. First, findings are presented regarding the characterization of molecular representation in pre-trained, fine-tuned, and projected CLM embeddings. Second, results are shown regarding the representation capabilities of summarization layers in AGGMs.

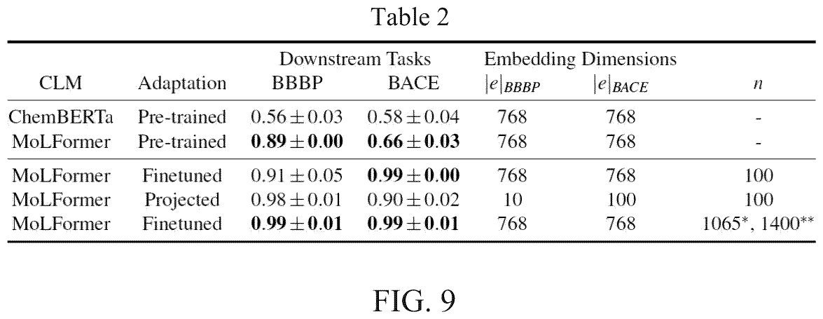

Representation in Chemical Language Model Embeddings: Initially, task-agnostic embeddings from both ChemBERTa and MoLFormer were compared, see Table 2 in FIG. 9. Since MoLFormer embeddings provided the best performance in both downstream tasks, only domain adaptation experiments were continued with this model.

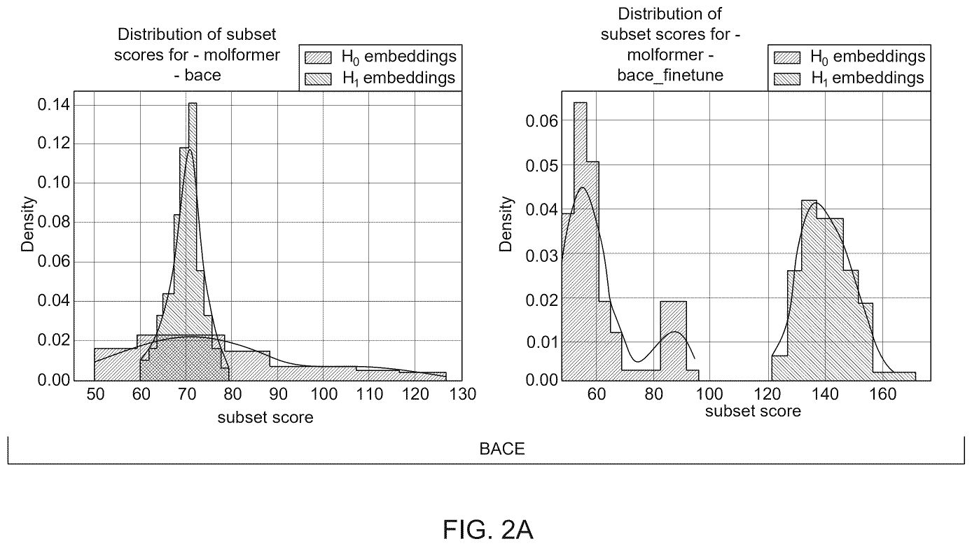

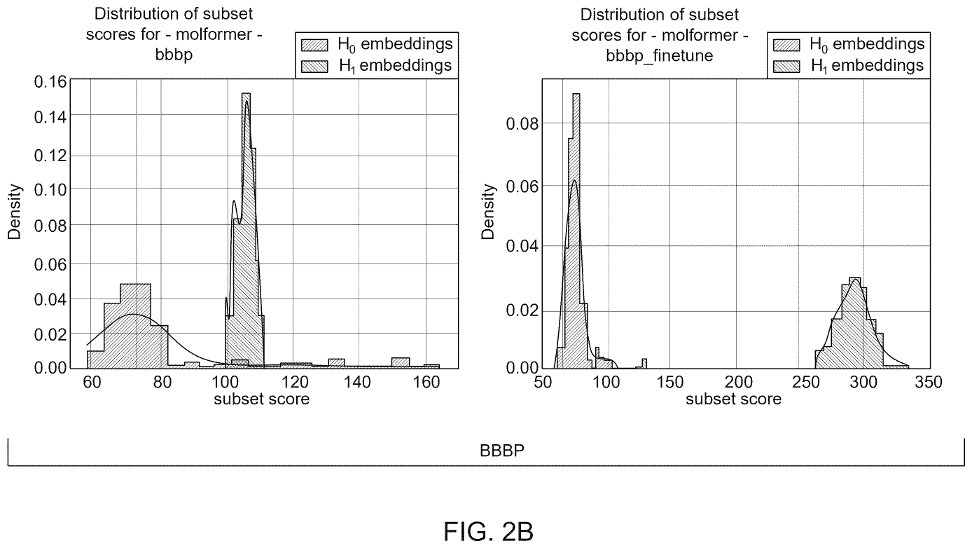

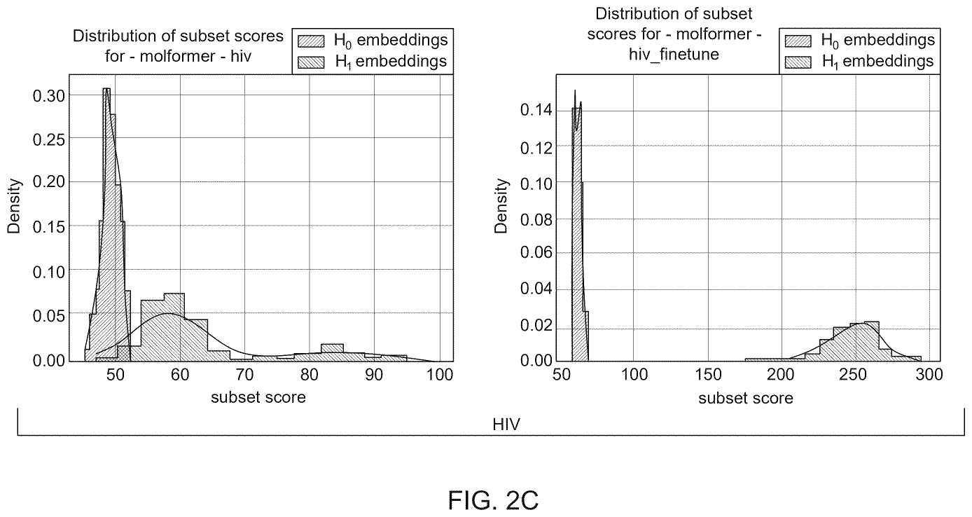

In FIGS. 2A through 2C, it can be observed how the detection power improves when scanning the finetuned embeddings compared to the task-agnostic version for each of the three binary classification tasks (HIV, BACE and BBBP). While the detection power increases in finetuned embeddings, the cardinality of elements needed to detect a given class is significantly smaller when scanning the finetuned representation (≈130 elements) compared to the task-agnostic (≈240 elements), which can be a step forward for detecting the subset of elements that are more likely to improve the quality of the representation for a given task via finetuning.

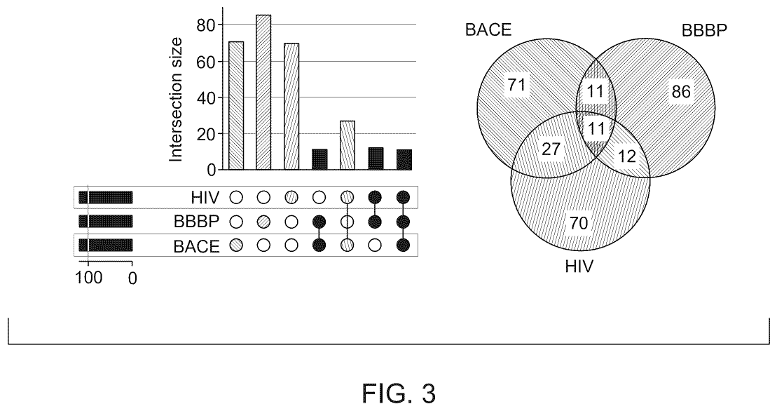

Even more importantly, it can be observed, in FIG. 3, when the most common elements are examined across all runs and then compared among the tasks (BBBP, BACE, and HIV), only 11 property-driven elements are shared in the three tasks, while ≈70-80 of those are unique to each task. Further, it can be seen that HIV and BACE share almost double the elements (27); both of these tasks involve enzyme inhibition, compared to the 11 and 12 nodes share with BBBP, which is associated with a fundamentally different mechanism. These results align with theoretical Information Bottleneck (IB) studies, which shows that IBs are desirable characteristics of optimal representations. IB builds the intuition that a representation should be maximally informative about a given target but contain no additional information about other domains. When the precision was averaged across 100 PDSS runs for task-agnostic and finetuned embeddings; it was seen that an approximate 0.23 improvement in the average precision (P). The task BACE has a P=0.62±0.04 for task-agnostic representation and P=0.88±0.04 for finetuned embedding. Similarly, BBBP P=0.78±0.04 for task-agnostic to P=0.99±0.01 while scanning the finetuned representation.

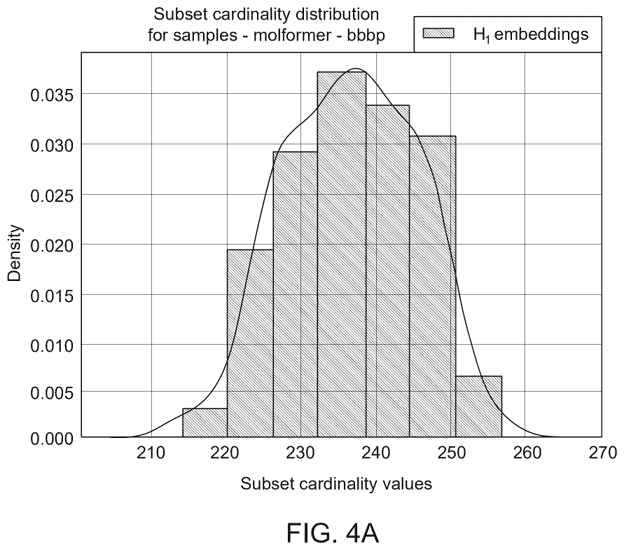

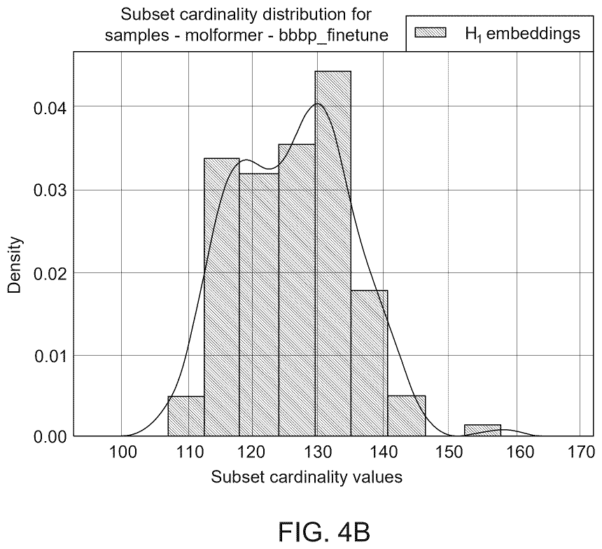

FIGS. 4A and 4B serve as a sanity check that there is a large portion of the same elements that get identified consistently across different test sets (the y-axis is the runs, and the x-axis refers to the location in the embedding e), which informs that those elements are relevant for the property-driven set across different candidates test sets. When the precision across 100 test runs were averaged for task-agnostic and fine-tuned embeddings; for both tasks, an approximately 0.23 improvement was seen in the average precision (P). P reflects the ability to label which molecule candidates in the test set belong to H₁, i.e., the property-driven set being of interest to be detected. The task BACE has a P=0.62±0.04 for task-agnostic representation and P=0.88±0.04 for fine-tuned embedding. Similarly, BBBP P=0.78±0.04 for task-agnostic to P=0.99±0.01 while scanning the fine-tuned representation.

Lastly, in a reduced data scenario when a small amount of target data for domain adaptation is available (n=100 in this experiment), the performance of projection methods and fine-tuning was compared (See Table 2 in FIG. 9). It can be observed that for the BBBP task, the projected embedding improves the Detection Power while requiring less computation and generating a reduced feature space than using the complete finetuned embedding. Nonetheless, it was observed that for the BACE task, reduced finetuning is still a better option than projection, hence the need for methods to select the best domain adaptation technique for a given task.

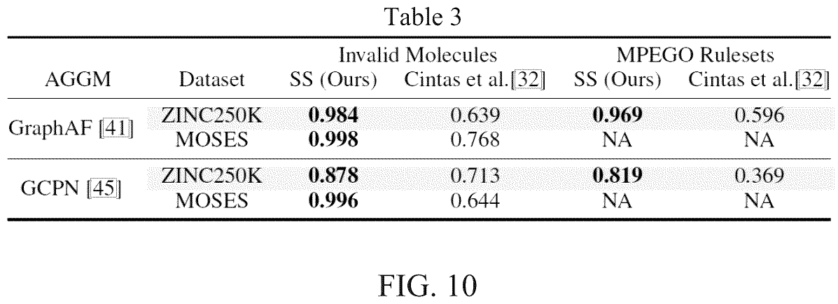

Representation in Graph Generative Models: Table 3, in FIG. 10, shows the detection power of the methods according to embodiments of the present disclosure for two different tasks across generative models and datasets. For all cases, it was observed that group-based scanning yields higher detection power than the baseline. It can be hypothesized that this is thanks to the unique ability to identify anomalous patterns activations across a group of candidate molecules to detect different tasks. The score distributions for the MPEGO rulesets can be seen in FIG. 7. It can be observed that the detection of power reduces compared to the validity task; this is partially because the expectation for the validity task is clearly divided from the alternative hypothesis, i.e., H₀ corresponds to invalid graph representations, while H₁ contains only valid graph representations. In the case of the MPEGO ruleset, both H₀ and H₁ contain valid representations. Particularly, H₁, contains valid representations with the given ruleset (shown in FIG. 7), which makes a more difficult detection problem. Furthermore, the impact of the H₀ definition was evaluated and it was shown that containing random valid and invalid representations as expectation yields higher detection capabilities. Nonetheless, a more conservative option is to build an expectation in only valid graph representations. Furthermore, from FIG. 7, it can be observed that the trained l_{Σ} from GraphAF shows a clear discrimination between activations that will generate a given ruleset compared to the rest of the possible properties combination. This confirms, from activation data, a potential bias of the model to be over-generating samples with those properties in the output space.

Evaluating which adaptation technique will work best for a new task. Adaptation techniques in reduced and complete data scenarios were assessed when the full dataset (n>1000) is available and when there is a small amount of target data (n=100). The performance of projection methods and finetuning was also contrasted (See Table 1, FIG. 8). It can be observed that for the BBBP task, the projected embedding improves the detection power while requiring less computation and generating a reduced feature space (|e|=10) than using the complete finetuned embedding. Nonetheless, it was observed that for the BACE task, finetuning with a smaller amount of target data is still a better option than projection and does not require access to the whole target data at finetuning time, hence the need for methods to select the best domain adaptation technique for a given task and data availability.

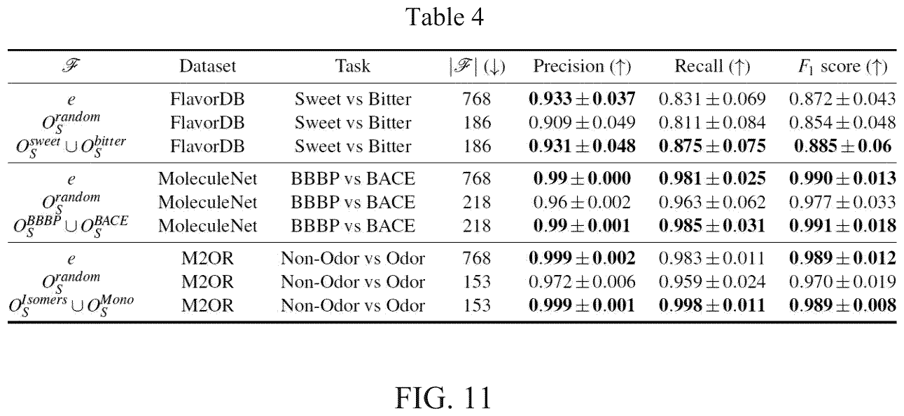

Evaluating the goodness of the subset of property-driven elements as predictors. Pre-trained embeddings were examined over new tasks, such as odor and flavor detection, to answer this question of goodness of the subset of property-driven elements as predictors. Odor molecules across different mixtures (Odor Mixture, Odor Isomers, Odor Mono) from M2OR26 and Sweet and Bitter flavors from FlavorDB27 were used. It was analyzed whether the subset of elements in the pre-trained embedding is useful for the downstream tasks. In Table 4, FIG. 11), it can be observed that while using less than 25% of the embedding, the same performance can be achieved as using the entire embedding for the downstream tasks and, in some cases, improve the overall metrics by 3 points.

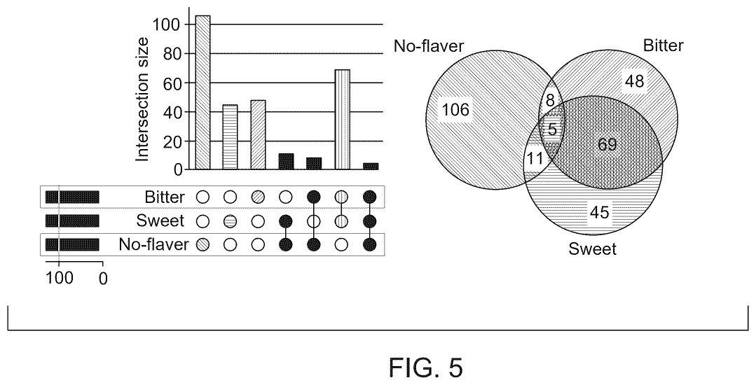

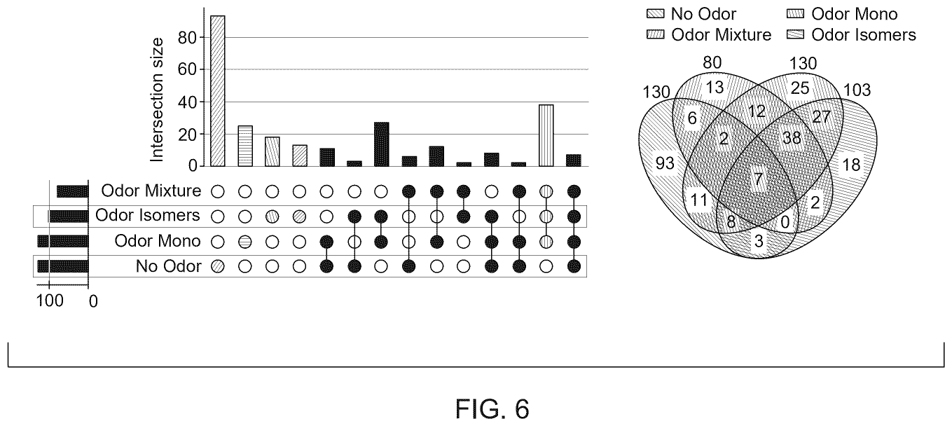

More interestingly, in FIGS. 5 and 6, it can be observed that property-driven elements found by methods according to embodiments of the present disclosure intersect the most with elements from similar tasks or properties; for example, the UpSet plot in FIG. 5 shows that the elements found for flavor representation molecules

$$\{ {O}_{S}^{bitter} ⋂ {O}_{s}^{sweet} \}$$

is six times greater than any other possible intersection between representation of no-flavor, sweet and bitter molecules. A similar pattern is visible in Odor molecules, where different types of odor representations share 38 elements

$$( \{ {O}_{S}^{isomers} S ⋂ {O}_{S}^{mono} ⋂ {O}_{S}^{mixture} \} ) ,$$

the highest intersection as shown in the UpSet plot. It can be hypothesized that this also reflects some semantic overlap of the information carried by these property-driven elements.

### Conclusions

With the surge of pre-trained deep learning models, the question of which representation is best for deployment is gaining importance to both ML researchers and practitioners. While chemical models have demonstrated enhanced performance in generative and predictive benchmarks, the interpretability and evaluation of the learned representations remain limited. Embodiments of the present disclosure can quantify the relative goodness of pre-trained representations in terms of task-specific information consolidation, as shown in FIGS. 2A and 2B and Table 2, in FIG. 9. Additionally, embodiments of the present disclosure can quantify the usefulness of different domain adaptation methods, as it was shown that projected embeddings seem to compress relevant information in compact feature vectors in reduced data settings for BBBP task, while fine-tuning showed to generate a more discriminative representation for both tasks (BACE and BBBP) when a larger amount of data is available (n>1000). Furthermore, it was observed that fine-tuned embeddings show information bottlenecks that are desirable for optimal representation, as the most common top ˜130 elements across tasks, only 8 property-driven elements are shared in the two tasks, while 122 of those are unique to each task. Lastly, is was shown how embodiments of the present disclosure can aid and evaluate the generative process, such as detecting in the activation space (lg) which elements tend to generate molecules with the same set of properties, as shown in FIG. 7 and Table 3 in FIG. 10.

The framework as described herein works across models with different architecture, inner representation types, and input features. A non-parametric group property-driven subset scanning approach can be used to analyze representation learning models and domain adaptation techniques.

While the above discussion focuses on models for the generation of molecules, it should be understood that the methods according to embodiments of the present invention may be used in other areas, such as Large Natural Language Models.

### Example Computing Platform

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

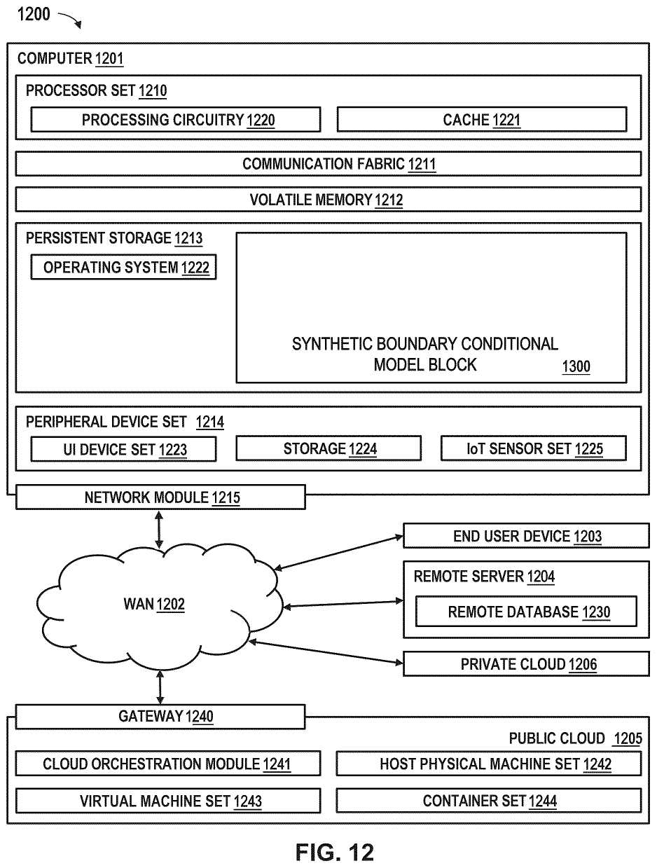

Referring to FIG. 12, computing environment 1200 includes an example of an environment for the execution of at least some of the computer code involved in performing the inventive methods, including a pre-trained model characterization and evaluation block 1300. In addition to block 1300, computing environment 1200 includes, for example, computer 1201, wide area network (WAN) 1202, end user device (EUD) 1203, remote server 1204, public cloud 1205, and private cloud 1206. In this embodiment, computer 1201 includes processor set 1210 (including processing circuitry 1220 and cache 1221), communication fabric 1211, volatile memory 1212, persistent storage 1213 (including operating system 1222 and block 1300, as identified above), peripheral device set 1214 (including user interface (UI) device set 1223, storage 1224, and Internet of Things (IoT) sensor set 1225), and network module 1215. Remote server 1204 includes remote database 1230. Public cloud 1205 includes gateway 1240, cloud orchestration module 1241, host physical machine set 1242, virtual machine set 1243, and container set 1244.

COMPUTER 1201 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 1230. As is well understood in the art of computer technology, and depending upon the technology, performance of a computer-implemented method may be distributed among multiple computers and/or between multiple locations. On the other hand, in this presentation of computing environment 1200, detailed discussion is focused on a single computer, specifically computer 1201, to keep the presentation as simple as possible. Computer 1201 may be located in a cloud, even though it is not shown in a cloud in FIG. 12. On the other hand, computer 1201 is not required to be in a cloud except to any extent as may be affirmatively indicated.

PROCESSOR SET 1210 includes one, or more, computer processors of any type now known or to be developed in the future. Processing circuitry 1220 may be distributed over multiple packages, for example, multiple, coordinated integrated circuit chips. Processing circuitry 1220 may implement multiple processor threads and/or multiple processor cores. Cache 1221 is memory that is located in the processor chip package(s) and is typically used for data or code that should be available for rapid access by the threads or cores running on processor set 1210. Cache memories are typically organized into multiple levels depending upon relative proximity to the processing circuitry. Alternatively, some, or all, of the cache for the processor set may be located “off chip.” In some computing environments, processor set 1210 may be designed for working with qubits and performing quantum computing.

Computer readable program instructions are typically loaded onto computer 1201 to cause a series of operational steps to be performed by processor set 1210 of computer 1201 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 1221 and the other storage media discussed below. The program instructions, and associated data, are accessed by processor set 1210 to control and direct performance of the inventive methods. In computing environment 1200, at least some of the instructions for performing the inventive methods may be stored in block 1300 in persistent storage 1213.

COMMUNICATION FABRIC 1211 is the signal conduction path that allows the various components of computer 1201 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 1212 is any type of volatile memory now known or to be developed in the future. Examples include dynamic type random access memory (RAM) or static type RAM. Typically, volatile memory 1212 is characterized by random access, but this is not required unless affirmatively indicated. In computer 1201, the volatile memory 1212 is located in a single package and is internal to computer 1201, but, alternatively or additionally, the volatile memory may be distributed over multiple packages and/or located externally with respect to computer 1201.

PERSISTENT STORAGE 1213 is any form of non-volatile storage for computers that is now known or to be developed in the future. The non-volatility of this storage means that the stored data is maintained regardless of whether power is being supplied to computer 1201 and/or directly to persistent storage 1213. Persistent storage 1213 may be a read only memory (ROM), but typically at least a portion of the persistent storage allows writing of data, deletion of data and re-writing of data. Some familiar forms of persistent storage include magnetic disks and solid state storage devices. Operating system 1222 may take several forms, such as various known proprietary operating systems or open source Portable Operating System Interface-type operating systems that employ a kernel. The code included in computing environment 1200 typically includes at least some of the computer code involved in performing the inventive methods.

PERIPHERAL DEVICE SET 1214 includes the set of peripheral devices of computer 1201. Data communication connections between the peripheral devices and the other components of computer 1201 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 1223 may include components such as a display screen, speaker, microphone, wearable devices (such as goggles and smart watches), keyboard, mouse, printer, touchpad, game controllers, and haptic devices. Storage 1224 is external storage, such as an external hard drive, or insertable storage, such as an SD card. Storage 1224 may be persistent and/or volatile. In some embodiments, storage 1224 may take the form of a quantum computing storage device for storing data in the form of qubits. In embodiments where computer 1201 is required to have a large amount of storage (for example, where computer 1201 locally stores and manages a large database) then this storage may be provided by peripheral storage devices designed for storing very large amounts of data, such as a storage area network (SAN) that is shared by multiple, geographically distributed computers. IoT sensor set 1225 is made up of sensors that can be used in Internet of Things applications. For example, one sensor may be a thermometer and another sensor may be a motion detector.

NETWORK MODULE 1215 is the collection of computer software, hardware, and firmware that allows computer 1201 to communicate with other computers through WAN 1202. Network module 1215 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 1215 are performed on the same physical hardware device. In other embodiments (for example, embodiments that utilize software-defined networking (SDN)), the control functions and the forwarding functions of network module 1215 are performed on physically separate devices, such that the control functions manage several different network hardware devices. Computer readable program instructions for performing the inventive methods can typically be downloaded to computer 1201 from an external computer or external storage device through a network adapter card or network interface included in network module 1215.

WAN 1202 is any wide area network (for example, the internet) capable of communicating computer data over non-local distances by any technology for communicating computer data, now known or to be developed in the future. In some embodiments, the WAN 1202 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 1203 is any computer system that is used and controlled by an end user (for example, a customer of an enterprise that operates computer 1201), and may take any of the forms discussed above in connection with computer 1201. EUD 1203 typically receives helpful and useful data from the operations of computer 1201. For example, in a hypothetical case where computer 1201 is designed to provide a recommendation to an end user, this recommendation would typically be communicated from network module 1215 of computer 1201 through WAN 1202 to EUD 1203. In this way, EUD 1203 can display, or otherwise present, the recommendation to an end user. In some embodiments, EUD 1203 may be a client device, such as thin client, heavy client, mainframe computer, desktop computer and so on.

REMOTE SERVER 1204 is any computer system that serves at least some data and/or functionality to computer 1201. Remote server 1204 may be controlled and used by the same entity that operates computer 1201. Remote server 1204 represents the machine(s) that collect and store helpful and useful data for use by other computers, such as computer 1201. For example, in a hypothetical case where computer 1201 is designed and programmed to provide a recommendation based on historical data, then this historical data may be provided to computer 1201 from remote database 1230 of remote server 1204.

PUBLIC CLOUD 1205 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 1205 is performed by the computer hardware and/or software of cloud orchestration module 1241. The computing resources provided by public cloud 1205 are typically implemented by virtual computing environments that run on various computers making up the computers of host physical machine set 1242, which is the universe of physical computers in and/or available to public cloud 1205. The virtual computing environments (VCEs) typically take the form of virtual machines from virtual machine set 1243 and/or containers from container set 1244. It is understood that these VCEs may be stored as images and may be transferred among and between the various physical machine hosts, either as images or after instantiation of the VCE. Cloud orchestration module 1241 manages the transfer and storage of images, deploys new instantiations of VCEs and manages active instantiations of VCE deployments. Gateway 1240 is the collection of computer software, hardware, and firmware that allows public cloud 1205 to communicate through WAN 1202.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 1206 is similar to public cloud 1205, except that the computing resources are only available for use by a single enterprise. While private cloud 1206 is depicted as being in communication with WAN 1202, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 1205 and private cloud 1206 are both part of a larger hybrid cloud.

### Conclusion

The descriptions of the various embodiments of the present teachings have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

While the foregoing has described what are considered to be the best state and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications, and variations that fall within the true scope of the present teachings.

The components, steps, features, objects, benefits, and advantages that have been discussed herein are merely illustrative. None of them, nor the discussions relating to them, are intended to limit the scope of protection. While various advantages have been discussed herein, it will be understood that not all embodiments necessarily include all advantages. Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

Numerous other embodiments are also contemplated. These include embodiments that have fewer, additional, and/or different components, steps, features, objects, benefits and advantages. These also include embodiments in which the components and/or steps are arranged and/or ordered differently.

Aspects of the present disclosure are described herein with reference to a flowchart illustration and/or block diagram of a method, apparatus (systems), and computer program products according to embodiments of the present disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of an appropriately configured computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The call-flow, flowchart, and block diagrams in the figures herein illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the blocks may occur out of order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While the foregoing has been described in conjunction with exemplary embodiments, it is understood that the term “exemplary” is merely meant as an example, rather than the best or optimal. Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments have more features than are expressly recited in each claim. Rather, as the following claims reflect, the inventive subject matter lies in less than all features of a single disclosed embodiment. Thus, the following claims are hereby incorporated into the Detailed Description, with each claim standing on its own as a separately claimed subject matter.

What is claimed is:

1. A computer-implemented method for characterizing and evaluating a pre-trained model, the method comprising:
training an unsupervised model using non-parametric property-driven subset scanning to provide a characterization of embeddings of the pre-trained model; and
using the characterizations to compute a metric to contrast different domain adaptation methods for the pre-trained model to improve an interpretability of deep molecular representations.

2. The computer-implemented method of claim 1, further comprising using the non-parametric property-driven subset scanning on language embeddings and node activations from summarization layers of the pre-trained model.

3. The computer-implemented method of claim 2, further comprising reducing a space of subsets through a linear-time subset scanning property while identifying a subset with a highest score.

4. The computer-implemented method of claim 1, wherein the metric is computed for a given task unseen by the pre-trained model.

5. The computer-implemented method of claim 1, wherein the training of the unsupervised model includes:
forming a distribution of expected activations at each element of an embedding vector;
scoring a group of samples in a test set by recording the embeddings induced by the group of test samples and comparing them to baseline embeddings created from the distribution of expected expectations to provide a p-value for each sample in the test set at each embedding; and
measuring a degree of anomalousness of each p-value.

6. The computer-implemented method of claim 1, further comprising using the characterizations to determine a wellness of the pre-trained model for a new task.

7. The computer-implemented method of claim 1, further comprising using the characterizations as element localization within the embeddings.

8. The computer-implemented method of claim 1, further comprising
receiving an input regarding computational constraints for running the pre-trained model; and
outputting a combination of the pre-trained model and adaptation techniques best for a new task and the computational constraints.

9. A computer-implemented method for characterizing and evaluating multiple pre-trained models, the method comprising:
training an unsupervised model using non-parametric property-driven subset scanning to provide a characterization of embeddings of each of the pre-trained models;
using the characterizations to compute a metric to rank each of the pre-trained models for a new task;
receiving an input regarding computational constraints for running the pre-trained model; and
outputting a combination of one of the pre-trained models and an adaptation technique best for the new task and the computational constraints.

10. The computer-implemented method of claim 9, further comprising using the non-parametric property-driven subset scanning methods on language embeddings and node activations from summarization layers of the pre-trained model.

11. The computer-implemented method of claim 10, further comprising reducing a space of subsets through a linear-time subset scanning property while identifying a subset with a highest score.

12. The computer-implemented method of claim 9, wherein the training of the unsupervised model includes:
forming a distribution of expected activations at each element of an embedding vector;
scoring a group of samples in a test set by recording the embeddings induced by the group of test samples and comparing them to baseline embeddings created from the distribution of expected expectations to provide a p-value for each sample in the test set at each embedding; and
measuring a degree of anomalousness of each p-value.

13. A computer program product for characterizing and evaluating a pre-trained model, the computer program product comprising a computer readable storage medium having program instructions embodied therewith, the program instructions executable by a computer to cause the computer to:
train an unsupervised model using non-parametric property-driven subset scanning to provide a characterization of embeddings of the pre-trained model; and
use the characterizations to compute a metric to contrast different domain adaptation methods for the pre-trained model.

14. The computer program product of claim 13, wherein the program instructions cause the computer to use the non-parametric property-driven subset scanning methods on language embeddings and node activations from summarization layers of the pre-trained model.

15. The computer program product of claim 14, wherein the program instructions cause the computer to reduce a space of subsets through a linear-time subset scanning property while identifying a subset with a highest score.

16. The computer program product of claim 13, wherein the metric is computed for a given task unseen by the pre-trained model.

17. The computer program product of claim 13, wherein the training of the unsupervised model includes:
forming a distribution of expected activations at each element of an embedding vector;
scoring a group of samples in a test set by recording the embeddings induced by the group of test samples and comparing them to baseline embeddings created from the distribution of expected expectations to provide a p-value for each sample in the test set at each embedding; and
measuring a degree of anomalousness of each p-value.

18. The computer program product of claim 13, wherein the program instructions cause the computer to use the characterizations to determine a wellness of the pre-trained model for a new task.

19. The computer program product of claim 13, wherein the program instructions cause the computer to use the characterizations as element localization within the embeddings.

20. The computer program product of claim 13, wherein the program instructions cause the computer to:
receive an input regarding computational constraints to run the pre-trained model; and
output a combination of the pre-trained model and adaptation techniques best for a new task and the computational constraints.
